(19) United States
(12) Patent Application Publication
(10) Pub. No.: US 2022/0064202 A1
(43) Pub. Date: Mar. 3, 2022

(54) GLYONIC LIQUIDS AND USES THEREOF

(71) Applicant: Arizona Board of Regents on behalf of the University of Arizona, Tucson, AZ (US)

(72) Inventors: Jeanne E. Pemberton, Tucson, AZ (US); Bhushan Deodhar, Tucson, AZ (US)

(73) Assignee: Arizona Board of Regents on behalf of the University of Arizona, Tucson, AZ (US)

(21) Appl. No.: 17/429,346

(22) PCT Filed: Feb. 9, 2020
(86) PCT No.: PCT/US2020/017387

§ 371 Date: Aug. 8, 2021

Related U.S. Application Data
(60) Provisional application No. 62/803,540, filed on Feb. 10, 2019.

(51) Int. Cl. C07H 3/06; H01M 8/14; B01D 53/14; C07H 3/04

(52) U.S. Cl. CPC C07H 3/06; H01M 8/144; B01D 53/1493; H01M 2300/0028; B01D 2252/30; H01M 2300/0002; C07H 3/04

(57) ABSTRACT

The present invention provides ionic liquids (ILs) comprising a carbohydrate anionic moiety and a cationic counter-ion moiety (Q⁺) and methods for producing and using the same. In one particular embodiment, the carbohydrate anionic moiety portion of ILs of the present invention is of the formula: (I) wherein G is selected from the group consisting of a monosaccharide, a disaccharide, a trisaccharide, and a derivative thereof; and L is a moiety selected from the group consisting of: (IIA) (IIB) wherein each of R^{a}, R^{b}, and R^{c} is independently hydrogen, C₁₋₁₈ alkyl, or C₂₋₂₀ mono- or di-unsaturated alkenyl; A^{TM} is —CO₂^{TM}, —PO₃H^{TM}, or —SO₃^{TM}; and each of * marked carbon atom is independently a chiral center when said carbon atom has four different groups attached thereto.

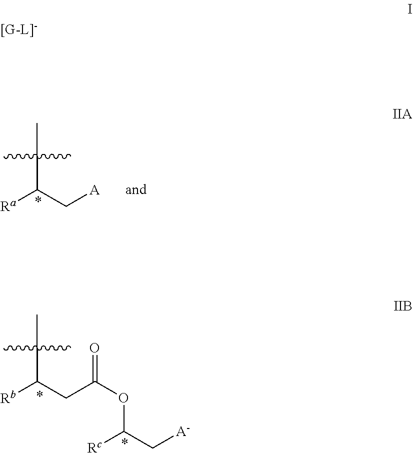

### CROSS-REFERENCE TO RELATED APPLICATIONS

This application claims the priority benefit of U.S. Provisional Application No. 62/803,540, filed Feb. 10, 2019, which is incorporated herein by reference in its entirety.

### STATEMENT REGARDING FEDERALLY FUNDED RESEARCH

This invention was made with government support under Grant No. CHE1339597, awarded by NSF. The government has certain rights in the invention.

### FIELD OF THE INVENTION

The present invention relates to ionic liquids comprising a carbohydrate anionic moiety and a cationic counter-ion moiety and methods for producing and using the same.

### BACKGROUND OF THE INVENTION

An ionic liquid (IL) is a salt in the liquid state at or close to room temperature (RT). Because ILs are salts, they typically have a very low vapor pressure. Ionic liquids have many potential applications including, but not limited to, as solvents, as electrically conducting fluids (electrolytes), as a catalyst in organic chemistry, in gas separation, etc. See, for example, Vekariya, R. L., in “A review of ionic liquids: Applications towards catalytic organic transformations,” J. Mol. Liq., 2017, 227, 44-60; Shang, D. et al., in “Ionic liquids in gas separation processing,” Current Opinion in Green and Sustainable Chemistry, 2017, 5, 74-81; A. S. Amarasekara, in “Acidic Ionic Liquids,” Chem. Rev. 2016, 116, 6133-6183; T. L. Greaves, C. J. Drummond in “Protic Ionic Liquids: Properties and Applications,” Chem. Rev. 2008, 108, 206-237; Z. Lei, C. Dai, B. Chen in “Gas Solubility in Ionic Liquids,” Chem. Rev. 2014, 114, 1289-1326; and J. Hulsbosch, D. E. De Vos, K. Binnemans, R. Ameloot in “Biobased Ionic Liquids: Solvents for a Green Processing Industry?,” ACS Sustainable Chem. Eng., 2016, 4, 2917-2931, all of which are incorporated herein by reference in their entirety.

Room temperature ionic liquids (RTILs) provide renewable and green attributes in a wide variety of applications including, but not limited to, as sustainable, green solvents to replace conventional volatile organic solvents, with a currently projected annual market of $3B. They also have the beneficial characteristics of electrical conductivity, non-flammability, and thermal and electrochemical stability.

Protic ionic liquids (PILs) are an important subclass of ionic liquids (ILs) that, by suitable molecular design, can be conferred with unique physicochemical properties similar to those of their aprotic counterparts, such as high ionic conductivity, low vapor pressure, high thermal stability, and low flammability. PILs have shown to be also useful in variety of applications, such as media for sustainable cotton dyeing, natural product extraction, biomass processing, protein and virus stabilization, biocatalysis, catalysis, gas separation, carbon dioxide capture, nanostructure formation, pharmaceutical applications, fuel processing, as capacitor electrolytes, as fuel cell electrolytes as well as other applications known to one skilled in the art. One particularly useful application for PILs is their use as fuel cell electrolytes due to the presence of labile protons in their cationic structure.

PILs can be formed by simple proton transfer from Brønsted acid to Brønsted base, leading to strong H-bonding. Ideally, proton transfer is complete in PILs, resulting in the presence of only cations and anions. In some cases, however, proton transfer is incomplete and can leave neutral acid and base species that limit the ionicity of the PIL. Nonetheless, PILs generally have higher values of conductivity and fluidity and lower melting points compared to aprotic ILs. One as-yet not fully realized aspiration of PILs is to develop systems in which the proton is freed from association with a specific ion, leading to superionic proton transport by a Grotthuss mechanism. Such materials have important applications in electrochemical systems that convert and store energy, thereby contributing to a more sustainable future.

While a wide variety of ILs and PILs are known, there still is a need for new ILs and PILs with other properties and advantages compared to conventional ILs and PILs.

### SUMMARY OF THE INVENTION

One aspect of the present invention provides an ionic liquid comprising a carbohydrate anionic moiety and a cationic counter-ion (Q⁺). While there have been multiple reports of carbohydrate-based anions in ILs, none of the conventional carbohydrate-based anions in IL are based on a tunable platform structure as disclosed herein. The carbohydrate based ILs of the present invention are sometimes referred to herein as glyonic liquids. The ionic liquid of the invention includes a conventional cationic counter-ion (Q⁺) such as, but not limited to, H⁺ (i.e., a protonated or a non-ionic form of carbohydrate anionic moiety of Formula I), imidazolium, pyridinium, pyrrolium, ammonium, iminium, phosphonium, sulfonium, an ester derivative of amino acid, choline, an ester derivative of betaine, acetylcholine, as well as other cationic counter-ions of ILs known to one skilled in the art.

In one particular embodiment, the carbohydrate anionic moiety portion of ILs of the present invention is of the formula:

[G-L]⁻ I

where G is selected from the group consisting of a monosaccharide, a disaccharide, a trisaccharide, and a derivative thereof; and L is a moiety of the formula:

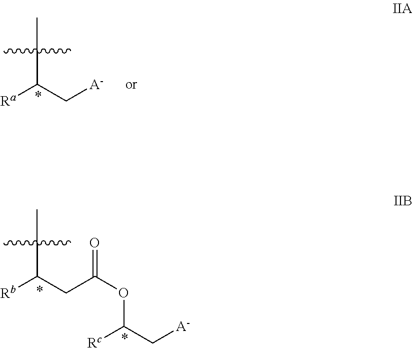

where each of R^{a}, R^{b}, and R^{c} is independently hydrogen, C₁₋₁₈ alkyl, or C₂₋₂₀ mono- or di-unsaturated alkenyl; A⁻ is —CO₂⁻, —PO₃H⁻, or —SO₃⁻, and each of * marked carbon atom is independently a chiral center when the carbon atom has four different groups attached thereto.

In some embodiments, said cationic counter-ion is selected from the group consisting of H⁺, imidazolium, pyridinium, pyrrolium, ammonium, iminium, phosphonium, sulfonium, an ester derivative of amino acid, choline, an ester derivative of betaine, and acetylcholine. It should be appreciated that when the cationic counter-ion (Q⁺) is proton, i.e., H⁺ or a protonated form of carbohydrate anion of Formula I, the resulting species is a neutral compound, whereas for other cationic counter-ions the resulting compound is electrically neutral but will contain an ionic bond between the carbohydrate anionic moiety and the cationic counter-ion. For the sake of brevity, however, both of these species are referred to herein as simply glyonic liquids or ionic liquids. Still in other embodiments, G is a monosaccharide or a derivative thereof. Yet in other embodiments, said monosaccharide is selected from the group consisting of glucose, galactose, rhamnose, arabinose, xylose, fucose, and glucosamine.

Still in other embodiments, G is a disaccharide or a derivative thereof. In one particular embodiment, said disaccharide is selected from the group consisting of lactose, maltose, melibiose, cellobiose, and rutinose. Yet in other embodiments, G is a trisaccharide or a derivative thereof. Yet in another embodiment, said trisaccharide is maltotriose.

In further embodiments, L is a moiety of the formula:

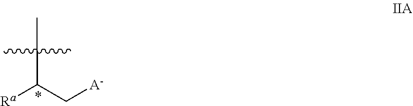

wherein A⁻, R^{a}, and * are as defined herein. In one particular embodiment, A⁻ is —COO⁻, R^{a} is a C₇-, C₉-, or C₁₁ alkyl, or C₉- or C₁₁-monounsaturated alkenyl, and * is an (R)-isomer.

Yet in other embodiments, L is a moiety of the formula:

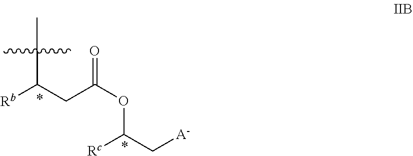

wherein A⁻, R^{b}, R^{c}, and * are those defined herein. In one particular embodiment, A⁻ is —COO⁻, and each of R^{b} and R^{c} is independently a C₇-, C₉-, or C₁₁-alkyl, or C₉- or C₁₁-monounsaturated alkenyl. Still in another embodiment, each of R^{b} and R^{c} is independently H, or C₁-, C₃-, C₅-, C₇-, C₉-, C₁₁-, C₁₃-alkyl, or C₉- or C₁₁-monounsaturated alkenyl.

Still in other embodiments, the melting point temperature of said ionic liquid is at most about 50° C.

Yet in further embodiments, said ionic liquid is a conductive non-crystalline semi-solid or solid at room temperature with a melting point temperature of at least 20° C.

Another aspect of the invention provides a superionic proton conductive material comprising an ionic liquid disclosed herein.

In some embodiments, the ionic liquid of the invention is solvated or hydrated.

Still another aspect of the invention provides a superionic proton conductor comprising an ionic liquid disclosed herein.

Yet in further aspect of the invention provides a fuel cell comprising an ionic liquid disclosed herein.

In another aspect of the invention, a battery is provided that comprises an ionic liquid disclosed herein. In some embodiments, the battery is a sodium or a lithium battery.

Yet another aspect of the invention provides a thermoelectric material comprising an ionic liquid disclosed herein.

In one particular embodiment, the ionic liquid is of the formula Qₓ⁺.[G-L]⁻, where x ranges from 0.5 to 1.

Another aspect of the invention provides a method for separating a gas from a gaseous mixture, said method comprising contacting the gaseous mixture with an ionic liquid of claim 1, thereby separating at least a portion of said gas from said gaseous mixture. In some embodiments, said gas comprises carbon dioxide, hydrogen sulfide, sulfur dioxide, methane, ethane, ethylene, propane, propylene, butane, 1-butene, oxygen, hydrogen, carbon monoxide, ammonia, water, Ar, Xe, CF₄, BF₃, AsH₃, or PH₃.

Other aspects of the invention provide a method of using ILs of the invention in variety of applications including those disclosed herein as well as other uses of ILs known to one skilled in the art.

### BRIEF DESCRIPTION OF THE DRAWINGS

FIG. 1 is a graph showing conductivity at 25° C., 70° C., and 90° C. as a function of added water content for the glyonic liquid (tBuNH₃)⁺.[Rha-C₁₀]⁻.

### DETAILED DESCRIPTION OF THE INVENTION

The present invention provides glyonic liquids and methods for producing and using the same in a wide variety of applications. As with all ionic liquids, the glyonic liquids of the invention include two components: an anionic moiety and a cationic counter-ion moiety (Q⁺). Together, these two moieties form a neutral substance. Unlike other liquids, which are neutral molecules, ionic liquids are made of ion pairs, i.e., when Q⁺ is not a proton (H⁺). Ionic liquids are also known in the art as liquid electrolytes, ionic melts, ionic fluids, fused salts, liquid salts, or ionic glasses.

Ionic liquids of the invention are comprised of a carbohydrate anionic moiety and a cationic counter-ion moiety (Q⁺). Accordingly, ionic liquids of the invention are also referred to herein as glyonic liquids due to the presence of carbohydrate anionic moiety. In one embodiment, the carbohydrate anionic moiety of glyonic liquids of the invention is of the formula:

[G-L]⁻ I

The glyonic liquids of the invention can also be represented by the formula Qₓ⁺.[G-L]_{y}⁻, where x and y are ratio of the cationic counter-ion moiety and the carbohydrate anionic moiety, respectively. As discussed herein, for the sake of brevity and clarity, it should be appreciated that compounds of the invention also include those where the cationic counter-ion is a proton, i.e., a protonated or a non-ionic form of Compound of Formula I. Such a compound is neutral compound and does not include an ionic bond. In carbohydrate anionic moiety of Formula I: G is selected from the group consisting of a monosaccharide, a disaccharide, a trisaccharide, and a derivative thereof; and L is a moiety selected from the group consisting of:

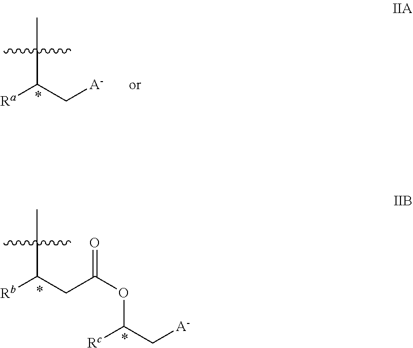

where each of R^{a}, R^{b}, and R^{c} is independently hydrogen, C₁₋₁₈ alkyl, or C₂₋₂₀ mono- or di-unsaturated alkenyl; A⁻ is —CO₂⁻, —PO₃H⁻, or —SO₃⁻, and each of * marked carbon atom is independently a chiral center when the carbon atom has four different groups attached thereto.

The asterisk (*) marks chiral center when that carbon atom has four different groups attached thereto. For example, when R^{a} is not hydrogen, then the carbon atom with the asterisk will be a chiral center. Similarly, when R^{b} or R^{c} is not hydrogen then the corresponding carbon atom will also be a chiral center. Unless otherwise specified, the chiral center can be an (S)-isomer or an (R)-isomer. The term “alkyl” refers to a saturated linear monovalent hydrocarbon moiety of one to eighteen (i.e., C₁₋₁₈) or a saturated branched monovalent hydrocarbon moiety of three to eighteen (i.e., C₃₋₁₈) carbon atoms. Exemplary alkyl group include, but are not limited to, methyl, ethyl, n-propyl, 2-propyl, tert-butyl, pentyl, undecyl, and the like.

The term “mono- or di-unsaturated alkenyl” refers to a linear or branched monovalent hydrocarbon moiety containing one or two carbon-carbon double bonds, respectively. Exemplary mono- or di-unsaturated alkenyls include, but are not limited to, ethenyl, propenyl, as well as other C₂₋₂₀ carbon chains having one or two carbon-carbon double bonds, respectively.

The term “sugar” and “carbohydrate” are used interchangeably herein and generally refers to a mono-, di-, and tri-saccharide. A carbohydrate is a biomolecule consisting of carbon (C), hydrogen (H) and oxygen (O) atoms, usually with a hydrogen-oxygen atom ratio of 2:1 (as in water) and thus with the empirical formula Cₘ(H₂O)ₙ (where m may be different from n). This formula holds true for monosaccharides. Some exceptions exist; for example, deoxyribose, a sugar component of DNA has the empirical formula C₅H₁₀O₄. The carbohydrates can be an aldose form or a ketose form. Carbohydrates can be obtained from natural sources, or can be biosynthesized. However, it should be appreciated that the source of carbohydrate does not limit the scope of invention. In fact, all sources of carbohydrates, either natural, synthetic, or combination thereof, are contemplated to be within the scope of the present invention.

The term “monosaccharide” refers to any type of hexose of the formula C₆H₁₂O₆ or a derivative thereof. The ring structure (i.e., ring type) of the monosaccharide can be a pyranose or a furanose. In addition, the monosaccharides can be an α- or β-anomer. Monosaccharide can be a ketonic monosaccharide (i.e., ketose), an aldehyde monosaccharide (i.e., aldose), or any type of hexose of the formula C₆H₁₂O₆ or a derivative thereof. Exemplary aldoses of the invention include, but are not limited to, allose, altrose, glucose, mannose, gulose, idose, galactose, talose, ribose, arabinose, xylose, lyxose, and derivatives thereof. Exemplary ketoses of the invention include, but are not limited to, psicose, fructose, sorbose, tagatose, ribulose, xylulose, and derivatives thereof. In one particular embodiment, the monosaccharide is selected from the group consisting of glucose, galactose, rhamnose, arabinose, xylose, fucose, and a thiol derivative thereof.

The term “disaccharide” refers to a carbohydrate composed of two monosaccharides. It is formed when two monosaccharides are covalently linked to form a dimer. The linkage can be a 1→2, 1→3, 1→4 or 1→6 etc. between the two monosaccharides. In addition, each of the monosaccharides can be independently an α- or β-anomer. Exemplary disaccharides that can be used in the present invention include, but are not limited to, sucrose, lactose, maltose, trehalose, cellobiose, lactulose, and chitobiose, etc. Each of the monosaccharides can independently be a ketonic monosaccharide (i.e., ketose), an aldehyde monosaccharide (i.e., aldose), or any type of hexose of the formula C₆H₁₂O₆ or a derivative thereof. Exemplary aldoses that can be used in preparing disaccharides of the invention include, but are not limited to, allose, altrose, glucose, mannose, gulose, idose, galactose, talose, ribose, arabinose, xylose, lyxose, and derivatives thereof. Exemplary ketoses that can be used in preparing disaccharides of the invention include, but are not limited to, psicose, fructose, sorbose, tagatose, ribulose, xylulose, and derivatives thereof. Each monosaccharide can also be independently an (L)-isomer or a (D)-isomer. In one particular embodiment, the disaccharide is selected from the group consisting of lactose, maltose, melibiose, cellobiose, rutinose, glucosamine, dirhamnose, and a derivative thereof, such as a thiol derivative.

The term “trisaccharide” refers to a carbohydrate composed of three monosaccharides. It is formed when three monosaccharides are covalently linked to form a trimer. The linkage can be a 1→2, 1→3, 1→4 or 1→6 etc. between the two monosaccharides. In addition, each of the monosaccharides can be independently an α- or β-anomer. Exemplary trisaccharides that can be used in the present invention include, but are not limited to, cellotriose, isomaltotriose, isopanose, laminaritriose, manninotriose, maltotriose, melezitose, nigerotriose, panose, raffinose, xylotriose, etc. Each of the monosaccharides can independently be a ketonic monosaccharide (i.e., ketose), an aldehyde monosaccharide (i.e., aldose), or any type of hexose of the formula C₆H₁₂O₆ or a derivative thereof. Exemplary aldoses that can be used in preparing trisaccharides of the invention include, but are not limited to, allose, altrose, glucose, mannose, gulose, idose, galactose, talose, ribose, arabinose, xylose, lyxose, and derivatives thereof. Exemplary ketoses that can be used in preparing trisaccharides of the invention include, but are not limited to, psicose, fructose, sorbose, tagatose, ribulose, xylulose, and derivatives thereof. Each saccharide can also be independently an (L)-isomer or a (D)-isomer. In one particular embodiment, the trisaccharide is maltotriose or a derivative thereof such as a thiol derivative.

As used herein the term “derivative” refers to a derivative of a saccharide in which one or more of the hydroxyl groups are replaced with hydrogen (e.g., 2-deoxy glucose, 5-deoxyglucose, etc.), an amine (e.g., amino sugars) or is replaced with a halogen, such as chloro, fluoro or iodo, (e.g., 5-fluoroglucose, 2-fluoroglucose, 5-chrologlucose, 2-chloroglucose, etc.). The term “derivative” also includes alkylated saccharides in which the hydroxy group is an alkoxy group (e.g., methoxy, ethoxy, etc.), as well as protected hydroxy group(s), such as acetylated, acetonides, etc., or otherwise modified using procedures known to one skilled in the art. Protecting groups for hydroxy groups of saccharides are well known to one skilled in the art. See, for example, T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, alkyl ethers, benzyl and trityl ethers, tetrahydropyranyl ethers, trialkylsilyl ethers and allyl ethers. In addition, the term “derivative” includes saccharides in which the —O— group that links “L” group in Formula I is replaced with another heteroatom such as —S— (i.e., thiol or a thiol derivative), —NR— (i.e., amine or an amine derivative, where R can be hydrogen, alkyl, or a nitrogen protecting group).

The “L” moiety in Formula I can be linked to G in either an α-configuration or a β-configuration. The composition can also include a mixture of α- and β-linked L moieties.

In one particular embodiment, G is rhamnose. In another embodiment, G is dirhamnose.

Still in another embodiment one or more * carbon is an (R)-isomer. In another embodiment, all * carbons are (R)-isomers.

Yet in another embodiment, L is of Formula IA. In some instances, R^{a} is a C₇, C₉, or C₁₁ alkyl. In another instances, R^{a} is C₉ or C₁₁ monounsaturated alkenyl.

In another embodiment, L is of Formula IIB. In some instances, each of R^{b} and R^{c} is independently a C₇, C₉, C₁₁ alkyl, or C₉ or C₁₁ monounsaturated alkenyl. Yet in other instances, R^{b} is H, or a 1-, 3-, 5-, 7-, 9-, 11-, 13-carbon alkyl chain, or C₉ or C₁₁ monounsaturated alkenyl; and R^{c} is H, or a 1-, 3-, 5-, 7-, 9-, 11-, 13-carbon alkyl chain, or C₉ or C₁₁ monounsaturated alkenyl. Still in some instances both * carbon are (R)-isomers. In other instances, both * carbon are (S)-isomers. Still in other instances one * carbon is an (R)-isomer and the other is an (S)-isomer.

In some embodiments, A⁻ is a carboxylate (i.e., —CO₂) moiety.

Yet in other embodiments, [G-L]⁻ is a single chain monorhamnolipid (i.e., α-L-rhamnopyranosyl-β-hydroxyalkanoates), a single chain dirhamnolipid (i.e., α-L-rhamnopyranosyl-α-L-rhamnopyranosyl-β-hydroxyalkanoates), a double chain monorhamnolipid (i.e., α-L-rhamnopyranosyl-β-hydroxyalkanoyl-β-hydroxyalkanoates) or a double chain dirhamnolipid (i.e. α-L-rhamnopyranosyl-α-L-rhamnopyranosyl-β-hydroxyalkanoyl-β-hydroxyalkanoates). As stated herein, all of the compounds of the invention can be synthetically made, produced biosynthetically by a microorganism, or can be produced by a combination thereof.

The cationic counter-ion moiety (Q⁺) can be any cationic counter-ion used in ILs that are known to one skilled in the art as well as a proton (i.e., H⁺, or simply a protonated form of carbohydrate anion of Formula I). They can be mixed with [G-L]⁻ in stoichiometric or substoichiometric amounts to fine tune properties. In one particular embodiment, the ionic liquid of the invention is of the formula: Qₓ+.[G-L]_{y}⁻, where x and y are ratio of the cationic counter-ion moiety and the carbohydrate anionic moiety, respectively. Typically, y is 1 and x can range from >0 to about 1, often x ranges from about ≥0.1 to about 1, more often from about ≥0.25 to about 1, still more often from about ≥0.5 to about 1, and most often from about ≥0.75 to about 1. When referring to a numerical value, the term “about” or “approximately” are used interchangeably herein and refer to being within an acceptable error range for the particular value as determined by one of ordinary skill in the art. Such a value determination will depend at least in part on how the value is measured or determined, e.g., the limitations of the measurement system, i.e., the degree of precision required for a particular purpose. For example, the term “about” can mean within 1 or more than 1 standard deviation, per the practice in the art. Alternatively, the term “about” when referring to a numerical value can mean±20%, typically ±10%, often ±5% and more often ±1% of the numerical value. In general, however, where particular values are described in the application and claims, unless otherwise stated, the term “about” means within an acceptable error range for the particular value, typically within one standard deviation.

Exemplary Q⁺ moieties that can be used in glyonic liquids of the invention include, but are not limited to, imidazolium, pyridinium, pyrrolium, ammonium, iminium, phosphonium, sulfonium, an ester derivative of amino acid, choline, an ester derivative of betaine, acetylcholine, as well as other cationic counter-ions of ILs known to one skilled in the art. Some specific Q⁺ moieties that can be used in glyonic liquids of the invention include the following particular cationic counter-ion moieties:

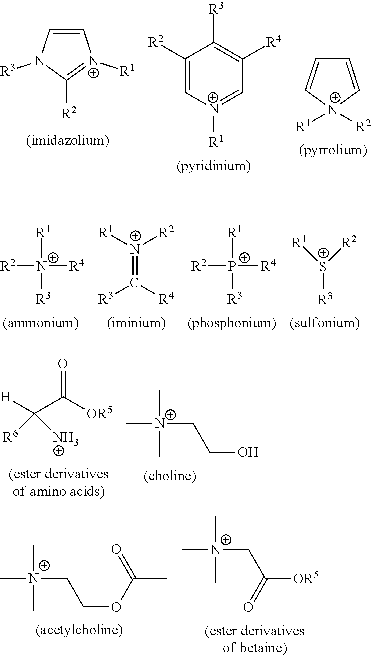

where each of R¹, R², R³, and R⁴ is independently H or C₁₋₁₀ alkyl; R⁵ is C₁₋₁₀ alkyl, typically C₁₋₆ alkyl, often C₁₋₄ alkyl; and R⁶ is a side-chain of amino acid.

As used herein the term “side-chain of amino acid” refers to any side-chains of both naturally occurring and synthetic amino acids. Exemplary side-chains of naturally occurring amino acids include the twenty-two (22) proteinogenic (“protein-building”) amino acids. In some embodiments, amino acid refers to the twenty-one (21) proteinogenic α-amino acids found in eukaryotes. In other embodiments, amino acid refers to and twenty (20) amino acids that are known to be used in encoding genetic code. However, it should be appreciated that the scope of the invention is not limited to twenty-two proteinogenic amino acids as any α-amino acids can be used in the present invention. It should also be noted that the stereochemistry of the amino acid can be either an (R)-isomer or an (S)-isomer, sometimes referred to as an (L)-isomer and a (D)-isomer.

In one particular embodiment, R⁶ is a side-chain of amino acid selected from the group consisting of glycine, alanine, valine, leucine, isoleucine, phenylalanine, proline, tryptophan, serine, threonine, methionine, cysteine, asparagine, and glutamine.

Yet in other embodiments, R⁵ is methyl, ethyl, propyl, isopropyl, tert-butyl, iso-butyl or butyl (i.e., n-butyl). Still in other embodiments, R⁵ is methyl or ethyl.

Still yet in other embodiments, R¹ is C₁₋₁₀ alkyl. Yet in other embodiments, each of R¹ and R² is independently C₁₋₁₀ alkyl.

Another aspect of the invention provides a composition comprising a glyonic liquid of the present invention. In some embodiments, the composition also includes aqueous solvent (i.e., water), non-aqueous solvent (e.g., organic solvent), or a mixture thereof. The composition can also optionally include salts, electrolytes, buffers, acids, bases, or other components that are used in various applications of ILs.

In some aspects of the invention, a glyonic liquid of the invention can be immobilized on a solid support. Such a solid support immobilized glyonic liquids can be used in gas separation, as a catalyst, as a conductive membrane in a fuel cell, etc. In general, glyonic liquids of the invention can be used in any and all applications that utilize ILs such as, but not limited to, as a catalyst in organic reactions, etc. In addition, glyonic liquids of the invention can also be used as a superionic proton conductive material.

In some embodiments, the glyonic liquids of the invention have a melting temperature of about 200° C. or less, typically about 100° C. or less, often about 60° C. or less, more often about 30° C. or less, and most often about 20° C. or less. In certain embodiments of the present invention, this characteristic, and the corresponding physical state of the glyonic liquid (i.e. solid, semi-solid (gel), or liquid) at different temperatures, can be tuned or tailored by inter alia selection of alkyl chain length in L. In general, longer chains lead to non-crystalline, conductive, semi-solid (gel) or solid forms at lower temperatures that have particular uses.

Another aspect of the invention provides a composition comprising a glyonic liquid of the present invention. In some embodiments, the glyonic liquid is present at lower temperatures (e.g., at about room temperature or 20° C. to 25° C.) as a noncrystalline semi-solid (gel) or solid. These semi-solid or solid glyonic liquid of the present invention have a melting point temperature of about 250° C. or less, typically about 200° C. or less, and most often at around 150° C. or less.

In some embodiments, glyonic liquids of this invention have a breakdown or degradation temperature, reported as the onset temperature (To) in thermogravimetric analysis (TGA), of about 250° C. or less, typically about 230° C. or less, and more often about 200° C. or less. To values from TGA for select glyonic liquids are given in Table 1. Further, the glyonic liquids of the present invention are thermally stable for at least tens of days at a temperature of about 200° C. or less, typically about 150° C. or less, often about 120° C. or less, more often about 70° C. or less, and most often about 40° C. or less. Specifically, the glyonic liquids [Glc-C₁₀]⁻(tBuNH₃)⁺ and [Xyl-C₁₀]⁻(tBuNH₃)⁺ are thermally stable for 30 days at a temperature of about 200° C. or less, typically about 150° C. or less, often about 120° C. or less, more often about 70° C. or less, and most often about 40° C. or less. As used herein, the term “thermally stable” means at least about 90%, typically at least about 95%, often at least about 98%, and most often at least about 99% of the glyonic liquid does not degrade at a given or stated temperature and time.

TABLE 1
Onset temperatures for selected glyonic
liquids from thermogravimetric analysis.

| Glyonic Liquid | Onset Temperature (T₀) |
|---|---|
| [Rha-C₁₀]⁻(tBuNH₃)⁺ | 225° C. |
| [Xyl-C₁₀]⁻(tBuNH₃)⁺ | 220° C. |
| [Glc-C₁₀]⁻(tBuNH₃)⁺ | 200° C. |
| [diRL-C₁₀C₁₀]⁻(tBuNH₃)⁺ | 230° C. |

In some embodiments, glyonic liquids of the invention have conductivity values that typically range from about 0.007 mS/cm to 100 mS/cm at temperatures between 25° C. and 100° C.

Another aspect of the invention provides superconductive glyonic liquids. Such superconductive glyonic liquids have properties similar to Nafion™ in which protons are able to move freely through the material, achieving higher ion mobility rates than diffusion rates. The rate of ion mobility can be determined by a variety of methods known to one skilled in the art. In some embodiments, the ion mobility rate is measured by conductivity.

Conductivity can be considerably enhanced in polar H-bonded “channels” that support Grotthuss superionic transport with the addition of small amounts of solvating solvents, either water or other protonic solvents (e.g., methanol, ethanol, isopropanol, t-butanol, etc.), to form the first solvation layer in a manner similar to Nafion.

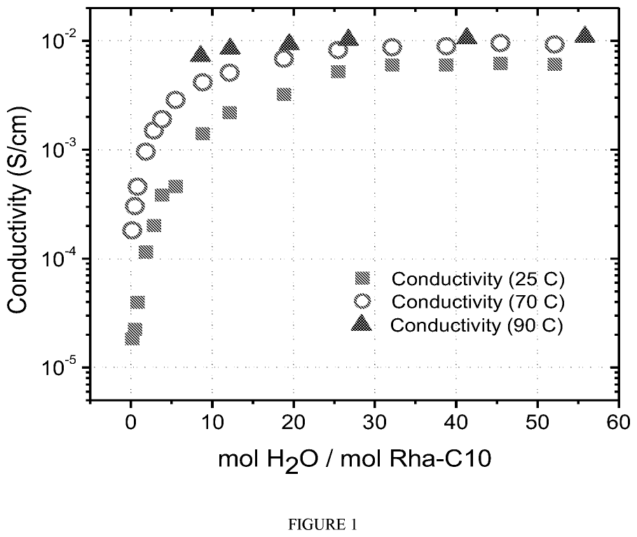

As one example of this effect, conductivity enhancement by Grotthuss superionic transport was seen in the glyonic liquid system [Rha-C₁₀]⁻(tBuNH₃)⁺ with the addition of water to fill the first hydration layer (FIG. 1). The increase in conductivity of over several orders of magnitude with hydrating waters is similar to that observed in Nafion.

Conventional superionic proton conductive materials are expensive to produce. Moreover, many superionic proton conductive materials are hazardous and are environmentally ill-suited for disposal. In contrast, glyonic liquid superconductors of the invention are significantly less costly to produce and are environmentally friendly in both production and use. Furthermore, superconductive glyonic liquids of the invention have a significant performance advantages, such as lower overpotential. As such, superconductive glyonic liquids of the invention can be used in a wide variety of applications such as fuel cells, batteries, thermoelectrics, wearable electronics, ion exchange materials, and other applications that require fast charge transfer.

Yet in other aspects of the invention, a glyonic liquid in its liquid, solid or semi-solid (gel) form can be copolymerized with a thermoelectric material such as PEDOT or other semiconducting polymers to enhance electrical properties. Such a glyonic liquid can be blended with relevant polymeric thermoelectric materials in different ratios to enhance the morphologies and electrical behavior. See for example, MacFarlane et al. in “Ionic liquids and their solid-state analogues as materials for energy generation and storage,” Nat. Rev. Mater., 2016, 1, article #15005 and references cited therein, all of which are incorporated by reference in their entirety.

In another embodiment, the glyonic liquid is used as electrolytes in lithium or sodium batteries in either its neat form or mixed with appropriate lithium or sodium salts, respectively. Alternatively, when used as lithium or sodium battery the cationic counter-ion Q⁺ is lithium ion or sodium ion, respectively. Such lithium or sodium glyonic liquid can be produced by cation exchange reaction where the cationic counter-ion Q⁺ is replaced or exchanged with lithium or sodium ions, respectively. Such a cationic exchange process is well known to one skilled in the art.

In one particular aspect of the invention, a glyonic liquid in its gel form, provides a conductive polymer for improving solid state lithium battery performance. In another application these ionic gels behave as supercapacitor electrolytes entrapped in a cross-linked polymer matrix. See for example, Taghavikish, et al. in “A Poly(ionic liquid) Gel Electrolyte for Efficient all Solid Electrochemical Double-Layer Capacitor,” Sci. Rep., 2018, 8, article #10918 and references cited therein, all of which are incorporated by reference in their entirety.

Glyonic liquids of the invention can also be used as the electrically conductive medium in biosensors for detection of multiple analytes of interest. In some embodiments, the glyonic liquids enhance the stability of biomarker probes such as proteins and enzymes used in wearable sensors. In another embodiment, glyonic liquids of the invention where the cationic counter-ion is choline provide new biocompatible conductors from inherently nonconductive polymeric materials. See for example, Munje et al. in “A new paradigm in sweat based wearable diagnostics biosensors using Room Temperature Ionic Liquids (RTILs),” Sci. Rep., 2017, 7, pp. 1950 and Noshadi et al. in “Engineering Biodegradable and Biocompatible Bio-ionic Liquid Conjugated Hydrogels with Tunable Conductivity and Mechanical Properties,” Sci. Rep., 2017, 7, pp. 4345, and references cited therein, all of which are incorporated by reference in their entirety.

Yet another aspect of the invention provides a fuel cell containing a glyonic liquid-based electrolyte or separator. In one particular embodiment, the fuel cell is an electric fuel cell.

Another aspect of the invention provides glyonic liquids as useful solvents for solubilization, harvesting, storage and crystallization of proteins. In one particular embodiment, the protein harvesting is in the form of protein crystals.

In another version of the invention, glyonic liquids of the invention in a semi-solid (e.g., gel) trapped in polymer matrices or supported on solid supports are utilized in environmental remediation to absorb organic contaminants from wastewater.

Yet another aspect of the invention provides a method for separating a gas from a gaseous mixture by contacting a gaseous mixture with a glyonic liquid of the invention. Use of ILs in separating gas are well known to one skilled in the art. See, for example, U.S. Pat. No. 6,579,343, issued to Brennecke et al., which is incorporated herein by reference in its entirety. Glyonic liquids are particularly suitable for separating carbon dioxide, hydrogen sulfide, sulfur dioxide, methane, ethane, ethylene, propane, propylene, butane, 1-butene, oxygen, hydrogen, carbon monoxide, ammonia, water, Ar, Xe, CF₄, BF₃, AsH₃, or PH₃ from a gaseous mixture.

Another aspect of the invention provides a carbohydrate-based deep eutectic solvent (DES). In one embodiment, the carbohydrate-based DES is a Type III deep eutectic solvent of the formula:

(P⁺X⁻).(G-L¹)_{z} III

where z is an integer 1 or 2; P⁺ is an organic cation; X⁻ is an anion, typically a Lewis base; G is as defined herein; and L¹ is a moiety selected from the group consisting of:

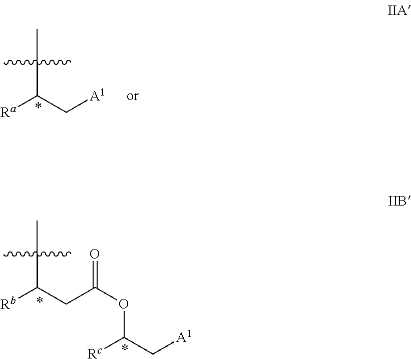

where *, R^{a}, R^{b}, and R^{c} are those defined herein; and A¹ is —COOH, —CONH₂, —OH, —SO₃H, or —PO₃H₂.

The terms “as defined above,” “as defined herein,” “those defined above,” and “those defined herein” when referring to a variable are used interchangeably and incorporate by reference the broad definition of the variable as well as any narrower definitions including any narrower definitions or preferred, more preferred and most preferred definitions, if any.

In one embodiment, P⁺ is a cationic counter-ion moiety Q⁺ as defined herein.

Still in another embodiment, X⁻ is a Lewis base anion. In some instances, X⁻ is a halide. In one particular embodiment, X⁻ is chloride.

In another embodiment, the carbohydrate-based DES is a Type IV deep eutectic solvent of the formula:

(MClₓ₋₁)⁺.(G-L¹)+(MClₓ₊₁)⁻ IV

where G and L¹ are those defined herein; MClₓ is a metal chloride; and x is an oxidation state of M.

MClₓ is any metal chloride that is used in DES Type IV. Generally, MClₓ is any metal chloride used by one skilled in the art for DES Type IV. In some embodiments, MClₓ is selected from the group consisting of nickel chloride, aluminum chloride, zinc chloride, copper chloride, silver chloride, tin chloride, and a hydrate thereof. The variable x refers to the oxidation state of metal M such that MClₓ is a neutral species, whereas MClₓ₋₁ is a cationic moiety and MClₓ₊₁ is an anionic moiety.

Glyonic liquids of the invention can also be used in electroplating or electrofinishing a metallic material. In some embodiments, the glyonic liquid used in electroplating a metallic material is a carbohydrate-based DES Type IV. In electroplating, typically the metallic material to be electroplated is contacted with a carbohydrate-based Type IV deep eutectic solvent of the invention under conditions sufficient to reduce anionic metal chloride species to form a film of metal M on the surface of the metallic material. Methods for electroplating using DES Type IV is well known to one skilled in the art. See, for example, Abbott et al. in “Electrofinishing of Metals Using Eutectic Based Ionic Liquids,” Trans. of the Inst. of Metal Finishing, 2008, 86(4), pp. 196-204 and references cited therein, all of which are incorporated by reference in their entirety.

In one embodiment, the metallic material is a metal. In general, any metal can be electroplated, e.g., coated with a metal M. In some embodiments, the metal M that is coated or electroplated onto the metallic material is selected from the group consisting of nickel, aluminum, zinc, copper, silver, and tin.

Conventional DES Type III and IV are often toxic, corrosive, and/or detrimental to environment, e.g., in production and/or disposal. In contrast, deep eutectic solvents of the invention (e.g., compounds of Formulas III and IV), are significantly less toxic and less corrosive. Moreover, DES of the present invention are renewable and green chemistry alternative for use in a wide variety of applications including, but not limited to, metal finishing for various precision and electronics manufacturing processes. In general, DES of the present invention can be used in any applications where Type III or Type IV DES is used.

The glyonic liquids of the invention, in particular G-L moiety of Formula I or G-L¹ moiety disclosed above, can be synthesized using the procedures as described in commonly assigned U.S. Pat. No. 9,499,575, issued Nov. 22, 2016, to Pemberton et al.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

### Synthesis of mRL—tert-Butyl Amine Glyonic Liquid

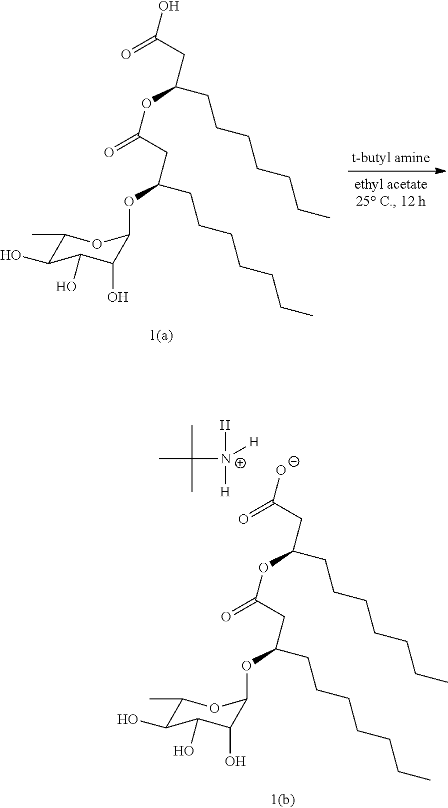

A flame-dried 10 mL round bottom flask under ambient conditions (e.g., room temperature at atmospheric pressure) was charged with ethyl acetate (4 mL) and 1 equiv (0.5 g, 1 mmol based on MW 504) of a biosynthesized (R,R) α-L-rhamnopyranosyl-β-hydroxyalkanoyl-β-hydroxyalkanoates (mRL) mixture harvested and purified from Pseudomonas aeruginaos ATCC 9027. Predominant congeners of this mixture typically include 80% (R,R) α-L-rhamnopyranosyl-β-hydroxydecanoyl-β-hydroxydecanoate, 10% (R,R) α-L-rhamnopyranosyl-β-hydroxyoctanoyl-β-hydroxydecanoates and/or (R,R) α-L-rhamnopyranosyl-β-hydroxydecanoyl-β-hydroxyoctanoates, 5% (R,R) α-L-rhamnopyranosyl-β-hydroxydecanoyl-β-hydroxydodecanoate and/or (R,R) α-L-rhamnopyranosyl-β-hydroxydodecanoyl-β-hydroxydecanoate, and 2% (R,R) α-L-rhamnopyranosyl-β-hydroxydecanoyl-β-hydroxydodec-5-enoate and/or (R,R) α-L-rhamnopyranosyl-β-hydroxydodec-5-enoyl-β-hydroxydecanoate). This congener mixture is represented above as (R,R) α-L-rhamnopyranosyl-β-hydroxydecanoyl-β-hydroxydecanoate (1(a)). To this solution was added dropwise 1 equiv of tert-butyl amine (0.11 mL, 1 mmol), and the reaction mixture was stirred at room temperature for 12 h. The mixture was then concentrated in vacuo to yield the desired glyonic liquid 1(b) as a pale yellow syrup in quantitative yield. The conductivity of 1(b) ranged from 0.007 mS/cm to 0.15 mS/cm at temperatures between at 25° C. and 90° C. In mixtures of 1(b) with small amounts of hydrating water, the conductivity ranged from 6 mS/cm to 10 mS/cm at temperatures between 25° C. and 70° C. ¹H NMR (500 MHz, chloroform-d) δ 5.38 (s, 1H), 4.84 (d, 1H), 4.27-4.25 (m, 1H), 3.81-3.78 (m, 2H), 3.77-3.72 (m, 2H), 2.40 (m, 4H), 1.65-1.45 (m, 2H), 1.36 (s, 9H), 1.27-1.25 (m, 23H), 0.92-0.84 (m, 6H). ¹³C NMR (125 MHz, chloroform-d) δ 171.99, 96.04, 75.16, 74.51, 73.85, 72.11, 69.56, 68.50, 51.16, 40.04, 34.88, 32.06, 31.96, 29.91, 29.71, 29.41, 29.36, 29.17, 28.80, 25.46, 24.86, 22.85, 22.82, 17.67, 14.27, 14.17.

### Synthesis of mRL—n-Butyl Amine Glyonic Liquid

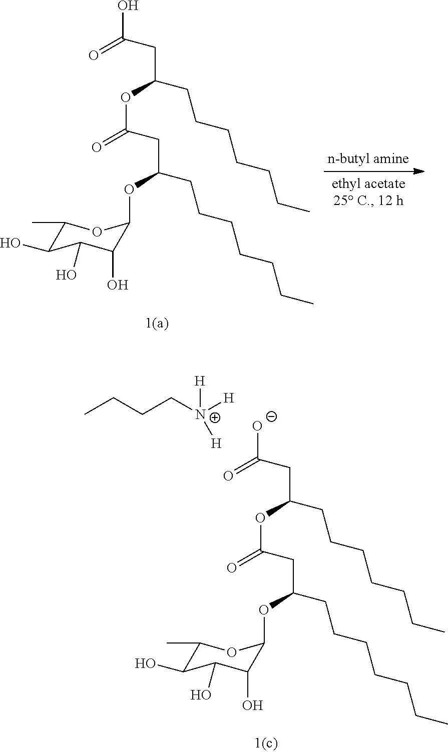

A flame-dried 10 mL round bottom flask under ambient conditions was charged with ethyl acetate (4 mL) and 1 equiv (0.5 g, 1 mmol based on MW 504) of a biosynthesized (R,R) α-L-rhamnopyranosyl-β-hydroxyalkanoyl-β-hydroxyalkanoates (mRL) mixture harvested and purified from Pseudomonas aeruginaos ATCC 9027. Predominant congeners of this mixture typically include 80% (R,R) α-L-rhamnopyranosyl-β-hydroxydecanoyl-β-hydroxydecanoate, 10% (R,R) α-L-rhamnopyranosyl-β-hydroxyoctanoyl-β-hydroxydecanoates and/or (R,R) α-L-rhamnopyranosyl-β-hydroxydecanoyl-β-hydroxyoctanoates, 5% (R,R) α-L-rhamnopyranosyl-β-hydroxydecanoyl-β-hydroxydodecanoate and/or (R,R) α-L-rhamnopyranosyl-β-hydroxydodecanoyl-β-hydroxydecanoate, and 2% (R,R) α-L-rhamnopyranosyl-β-hydroxydecanoyl-β-hydroxydodec-5-enoate and/or (R,R) α-L-rhamnopyranosyl-β-hydroxydodec-5-enoyl-β-hydroxydecanoate). This congener mixture is represented above as (R,R) α-L-rhamnopyranosyl-β-hydroxydecanoyl-β-hydroxydecanoate (1(a)). To this solution was added dropwise 1 equiv of n-butyl amine (0.1 mL, 1 mmol), and the reaction mixture was stirred at room temperature for 12 h. The mixture was then concentrated in vacuo to yield the desired glyonic liquid 1(c) as a pale yellow syrup in quantitative yield. ¹H NMR (500 MHz, Chloroform-d) δ 5.40 (d, 1H), 4.26 (tt, 4H), 3.86-3.65 (m, 2H), 3.25 (t, 4H), 2.58-2.36 (m, 4H), 1.64 (tt, 2H), 1.55-1.43 (m, 2H), 1.42-1.34 (m, 2H), 1.29 (m, 3H), 1.26 (m, 22H), 0.90-0.83 (m, 9H). ¹³C NMR (125 MHz, Chloroform-d) δ 175.35, 168.22, 100.12, 75.16, 73.49, 72.76, 71.98, 71.17, 71.06, 70.36, 68.35, 40.40, 39.75, 38.89, 34.85, 32.05, 31.65, 29.90, 29.83, 29.67, 29.40, 29.34, 25.34, 24.73, 22.37, 19.28, 16.86, 14.25, 13.73.

### Synthesis of mRL—Ammonium Glyonic Liquid

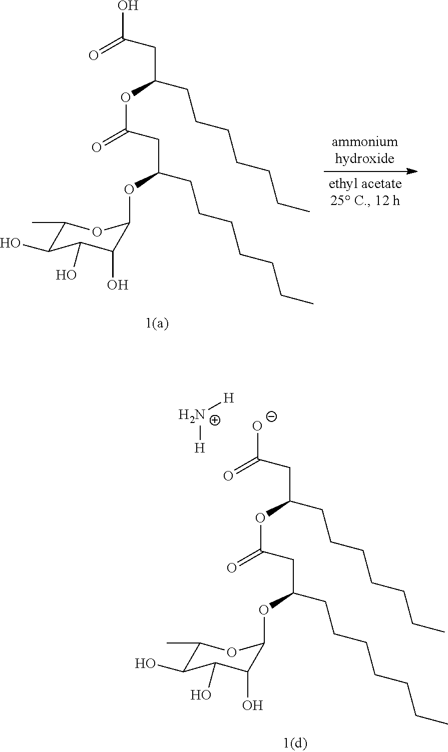 [SMILES: CCCCCCC[C@H](CC(=O)O)OC(=O)C[C@@H](CCCCCCC)OC1OC(C)C(O)C(O)C1O ; CCCCCCC[C@H](CC(=O)[O-])OC(=O)C[C@@H](CCCCCCC)OC1OC(C)C(O)C(O)C1O ; [NH4+]]

A flame-dried 10 mL round bottom flask under ambient conditions is charged with ethyl acetate (4 mL) and 1 equiv (0.5 g, 1 mmol based on MW 504) of a biosynthesized (R,R) α-L-rhamnopyranosyl-β-hydroxyalkanoyl-β-hydroxyalkanoates (mRL) mixture harvested and purified from Pseudomonas aeruginaos ATCC 9027. Predominant congeners of this mixture typically include 80% (R,R) α-L-rhamnopyranosyl-β-hydroxydecanoyl-β-hydroxydecanoate, 10% (R,R) α-L-rhamnopyranosyl-β-hydroxyoctanoyl-β-hydroxydecanoates and/or (R,R) α-L-rhamnopyranosyl-β-hydroxydecanoyl-β-hydroxyoctanoates, 5% (R,R) α-L-rhamnopyranosyl-β-hydroxydecanoyl-β-hydroxydodecanoate and/or (R,R) α-L-rhamnopyranosyl-β-hydroxydodecanoyl-β-hydroxydecanoate, and 2% (R,R) α-L-rhamnopyranosyl-β-hydroxydecanoyl-β-hydroxydodec-5-enoate and/or (R,R) α-L-rhamnopyranosyl-β-hydroxydodec-5-enoyl-β-hydroxydecanoate). This congener mixture is represented above as (R,R) α-L-rhamnopyranosyl-β-hydroxydecanoyl-β-hydroxydecanoate (1(a)). To this solution is added dropwise 1 equiv of ammonium hydroxide (0.04 mL, 1 mmol), and the reaction mixture is stirred at room temperature for 12 h. The mixture is then concentrated in vacuo to yield the desired glyonic liquid 1(d) as a syrup in quantitative yield.

### Synthesis of Rha-C₁₀-C₁₀—tert-Butyl Amine Glyonic Liquid

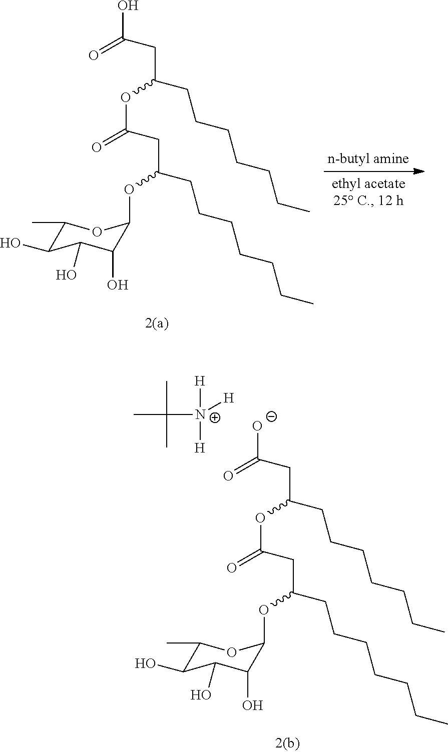

To a flame-dried 10 mL round bottom flask under ambient conditions was added 1 equiv of a diastereomeric mixture of α-L-rhamnopyranosyl-β-hydroxydecanoyl-β-hydroxydecanoate (2(a)) comprised of approximately equivalent amounts of the (R,R), (R,S), (S,R), and (S,S) diastereomers (0.5 g, 1 mmol) and ethyl acetate (4 mL). To the resulting solution was added dropwise 5 equiv of tert-butyl amine (0.52 mL, 5 mmol), and the reaction mixture was stirred at room temperature for 12 h. The mixture was then concentrated in vacuo to yield the desired glyonic liquid 2(b) as a pale yellow syrup in quantitative yield. ¹H NMR (499 MHz, chloroform-d) δ 5.32 (s, 1H), 4.88 (d, 1H), 4.26-4.22 (m, 1H), 3.83-3.75 (m, 2H), 3.68-3.52 (m, 2H), 2.40 (m, 4H), 1.58-1.45 (m, 2H), 1.28 (m, 9H), 1.26-1.20 (m, 23H), 0.90-0.85 (m, 6H). ¹³C NMR (125 MHz, chloroform-d) δ 179.90, 177.67, 177.41, 176.85, 173.04, 171.71, 171.17, 170.62, 101.90, 97.50, 95.34, 73.94, 73.57, 73.38, 73.33, 72.82, 72.59, 72.35, 72.27, 72.01, 71.74, 71.65, 71.00, 70.91, 69.01, 68.86, 68.50, 44.14, 43.00, 41.76, 39.71, 36.68, 35.59, 34.06, 32.28, 31.85, 31.80, 30.71, 30.35, 29.77, 29.64, 29.27, 28.10, 25.36, 25.23, 25.10, 24.58, 21.34, 17.74, 17.56, 13.65.

### Synthesis of Rha-C₁₀—tert-Butyl Amine Glyonic Liquid

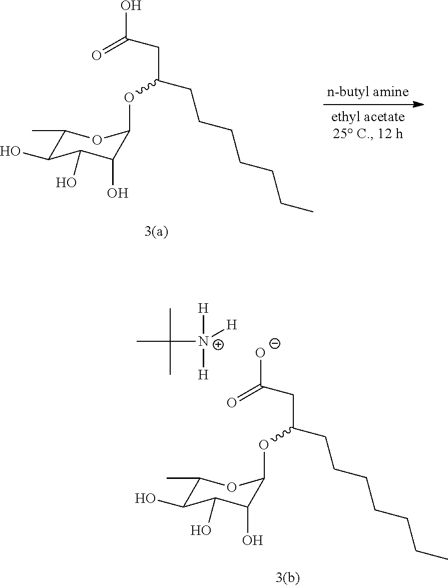

To a flame-dried 10 mL round bottom flask under ambient conditions was added 1 equiv of a diastereomeric mixture of α-L-rhamnose 3-hydroxydecanoic acid (3(a)) comprised of approximately equivalent amounts of the R and S diastereomers (0.5 g, 1 mmol) and ethyl acetate (4 mL). To the resulting solution was added dropwise 1 equiv of tert-butyl amine (0.15 mL, 1 mmol), and the reaction mixture was stirred at room temperature for 12 h. The mixture was then concentrated in vacuo to yield the desired glyonic liquid 3(b) as a pale yellow syrup in quantitative yield. The conductivity of 3(b) ranged from 0.008 mS/cm to 0.19 mS/cm at temperatures between 22° C. and 90° C. In mixtures of 3(b) with small amounts of hydrating water, the conductivity ranged from 6 mS/cm to 11 mS/cm at temperatures between 25° C. and 90° C. ¹H NMR (500 MHz, methanol-d₄) δ 4.62 (dd, 1H), 3.87-3.80 (m, 1H), 3.59-3.39 (m, 2H), 3.19-3.14 (m, 2H), 2.32-1.98 (m, 2H), 1.39-1.27 (m, 4H), 1.16 (s, 9H), 1.14-1.09 (m, 10H), 1.08-1.07 (d, 3H), 0.67 (t, 3H). ¹³C NMR (125 MHz, methanol-d₄) δ 185.96, 179.83, 179.78, 102.58, 98.94, 78.30, 76.57, 74.71, 73.91, 72.70, 72.37, 70.46, 69.78, 54.40, 45.71, 44.61, 36.34, 34.88, 33.00, 30.86, 30.74, 30.37, 28.46, 26.55, 25.98, 24.42, 18.06, 18.02, 13.37.

### Synthesis of Rha-C₁₀—n-Butyl Amine Glyonic Liquid

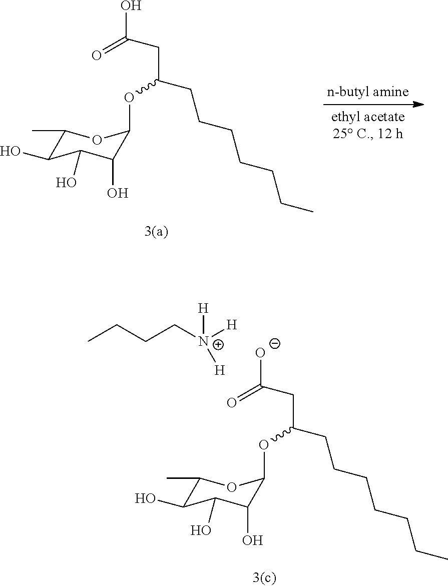

To a flame-dried 10 mL round bottom flask under ambient conditions is added 1 equiv of a diastereomeric mixture of α-L-rhamnose 3-hydroxydecanoic acid (3(a)) comprised of approximately equivalent amounts of the R and S diastereomers (0.5 g, 1 mmol) and ethyl acetate (4 mL). To the resulting solution is added dropwise 1 equiv of ammonium hydroxide (0.15 mL, 1 mmol), and the reaction mixture is stirred at room temperature for 12 h. The mixture is then concentrated in vacuo to yield the desired glyonic liquid 3(c) as a syrup in quantitative yield.

### Synthesis of Rha-C₁₀—Ammonium Glyonic Liquid

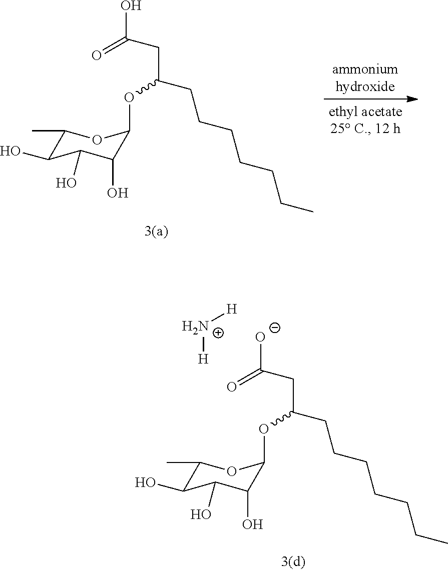 [SMILES: CCCCCCCC(CC(=O)O)OC1OC(C)C(O)C(O)C1O ; CCCCCCCC(CC(=O)[O-])OC1OC(C)C(O)C(O)C1O ; [NH4+]]

To a flame-dried m round bottom flask under ambient conditions was added 1 equiv of a diastereomeric mixture of α-L-rhamnose 3-hydroxydecanoic acid (3(a)) comprised of approximately equivalent amounts of the R and S diastereomers (0.5 g, 1 mmol) and ethyl acetate (4 mL). To the resulting solution was added dropwise 1 equiv of ammonium hydroxide (0.06 mL, 1 mmol), and the reaction mixture was stirred at room temperature for 12 h. The mixture was then concentrated in vacuo to yield the desired glyonic liquid 3(d) as a pale yellow syrup in quantitative yield. ¹H NMR (500 MHz, methanol-d₄) δ 4.80-4.73 (dd, 1H), 3.98 (m, 1H), 3.71-3.55 (m, 2H), 3.41-3.23 (m, 2H), 2.45-2.18 (m, 2H), 1.51-1.1.29 (m, 4H), 1.23-1.19 (m, 10H), 1.13-1.10 (d, 3H), 0.82 (t, 3H). ¹³C NMR (125 MHz, methanol-d₄) δ 179.53, 179.52, 101.53, 100.04, 77.82, 76.41, 74.23, 74.02, 72.42, 70.13, 69.93, 49.85, 49.00, 44.36, 43.98, 36.72, 34.42, 33.01, 32.97, 30.83, 30.74, 30.38, 26.50, 25.94, 23.69, 23.67, 17.91, 14.38.

### Synthesis of Xylose-C₁₀—tert-Butyl Amine Glyonic Liquid

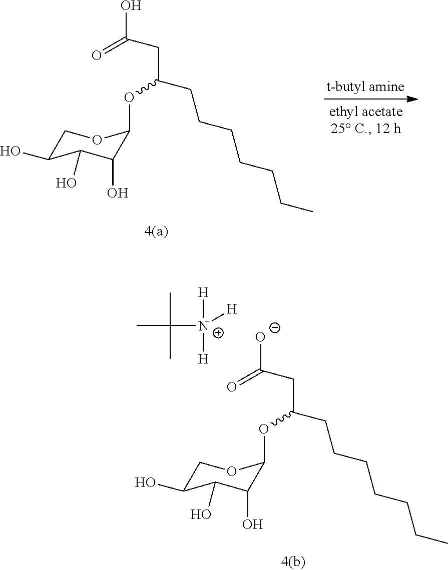

To a flame-dried 10 mL round bottom flask under ambient conditions was added 1 equiv of a diastereomeric mixture of xylose 3-hydroxydecanoic acid (4(a)) comprised of approximately equivalent amounts of the α and β anomers and R and S diastereomers (0.5 g, 1 mmol) and ethyl acetate (4 mL). To the resulting solution was added dropwise 1 equiv of tert-butyl amine (0.16 mL, 1 mmol), and the reaction mixture was stirred at room temperature for 12 h. The mixture was then concentrated in vacuo to yield the desired glyonic liquid 4(b) as a pale yellow syrup in quantitative yield. ¹H NMR (500 MHz, methanol-d₄) δ 5.10 (dd, 1H), 4.26 (d, 1H), 4.18-3.85 (m, 2H), 3.80-3.73 (m, 1H), 3.46-3.35 (m, 1H), 2.60-2.17 (m, 2H), 1.59-1.45 (m, 2H), 1.28-1.22 (m, 19H), 0.84 (t, 3H). ¹³C NMR (125 MHz, methanol-d₄) δ 180.40, 180.28, 110.50, 107.97, 105.11, 104.72, 104.50, 102.58, 100.76, 100.46, 84.22, 83.99, 82.58, 81.71, 79.18, 78.01, 76.83, 76.72, 76.70, 75.42, 74.97, 74.50, 73.94, 73.79, 72.44, 71.35, 71.25, 67.82, 66.72, 63.48, 62.53, 51.84, 49.85, 45.92, 44.33, 43.73, 36.67, 36.44, 36.30, 33.03, 30.92, 30.87, 30.70, 30.39, 30.35, 28.40, 26.48, 26.35, 26.26, 26.11, 23.68, 22.97, 14.40.

### Synthesis of Glucose-C₁₀—tert-Butyl Amine Glyonic Liquid

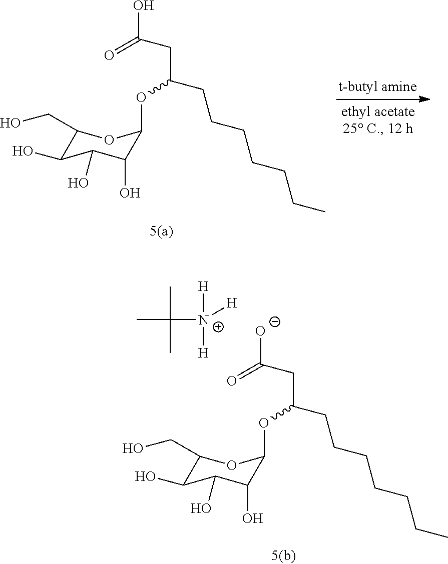

To a flame-dried 10 mL round bottom flask under ambient conditions was added 1 equiv of a diastereomeric mixture of glucose 3-hydroxydecanoic acid (5(a)) comprised of approximately equivalent amounts of the α and β anomers and R and S diastereomers (0.5 g, 1 mmol) and ethyl acetate (4 mL). To the resulting solution was added dropwise 1 equiv of tert-butyl amine (0.15 mL, 1 mmol), and the reaction mixture was stirred at room temperature for 12 h. The mixture was then concentrated in vacuo to yield the desired glyonic liquid 5(b) as a pale yellow syrup in quantitative yield. ¹H NMR (500 MHz, methanol-d₄) δ 4.34 (d, 2H), 4.09-4.00 (m, 1H), 3.82-3.75 (m, 1H), 3.36-3.30 (m, 2H), 3.22-3.15 (m, 1H), 3.15-3.08 (m, 1H), 2.52-2.23 (m, 2H), 1.31-1.28 (m, 10H), 1.27 (s, 9H), 0.85 (t, 3H). ¹³C NMR (126 MHz, methanol-d₄) δ 181.57, 180.42, 111.01, 103.88, 103.66, 102.94, 79.42, 79.31, 78.14, 78.10, 77.99, 75.59, 75.37, 71.88, 71.67, 67.84, 63.07, 62.90, 52.18, 46.31, 44.61, 36.89, 36.40, 36.21, 33.60, 31.44, 30.80, 30.41, 30.39, 28.55, 27.06, 26.40, 26.33, 25.89, 24.75, 23.69, 15.21, 14.39.

### Synthesis of Cellobiose-C₁₀C₁₀—Propyl Amine Glyonic Liquid

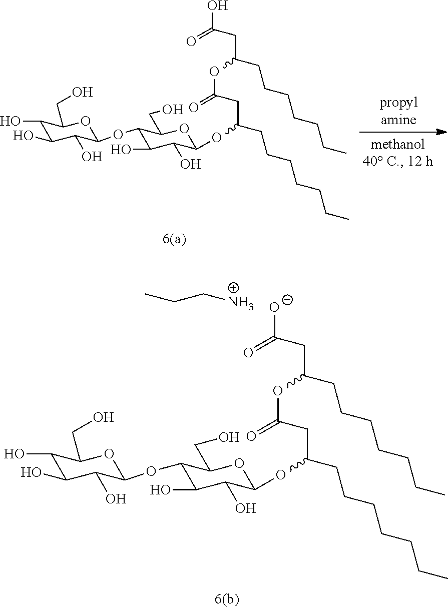

To a flame-dried 10 mL round bottom flask under ambient conditions is added 1 equiv of a diastereomeric mixture of 4-O-β-D-glucopyranosyl-D-glucose-β-hydroxydecanoyl-β-hydroxydecanoate (6(a)) comprised of approximately equivalent amounts of the (R,R), (R,S), (S,R), and (S,S) diastereomers (0.5 g, 0.73 mmol) and methanol (4 mL). To the resulting solution is added dropwise 5 equiv of propyl amine (0.30 mL, 3.66 mmol), and the reaction mixture is stirred in a sand bath at 40° C. for 12 h. The mixture is then concentrated in vacuo to yield the desired glyonic liquid 6(b) as a syrup in quantitative yield.

### Synthesis of Maltotriose-C₁₀C₁₀—Propyl Amine Glyonic Liquid

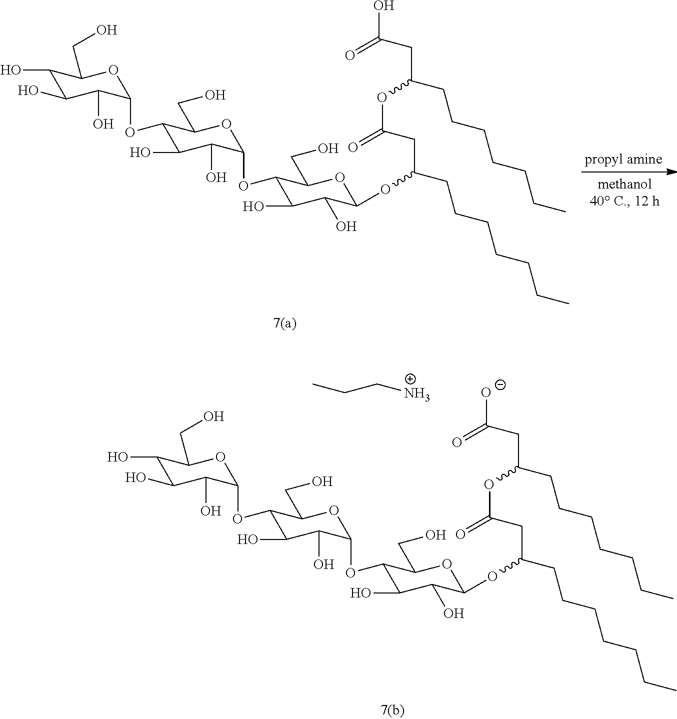

To a flame-dried 10 mL round bottom flask under ambient conditions is added 1 equiv of a diastereomeric mixture of O-α-DD-glucopyranosyl-(1-4)-O-α-D-glucopyranosyl-(1-4)-D-glucose-1-hydroxydecanoyl-β-hydroxydecanoate (7(a)) comprised of approximately equivalent amounts of the (R,R), (R,S), (S,R), and (S,S) diastereomers (0.5 g, 0.60 mmol) and methanol (4 mL). To the resulting solution is added dropwise 5 equiv of propyl amine (0.24 mL, 3.00 mmol), and the reaction mixture is stirred in a sand bath at 40° C. for 12 h. The mixture is then concentrated in vacuo to yield the desired glyonic liquid 7(b) as a syrup in quantitative yield.

### Synthesis of diRL-C₁₀-C₁₀—tert-Butyl Amine Glyonic Liquid

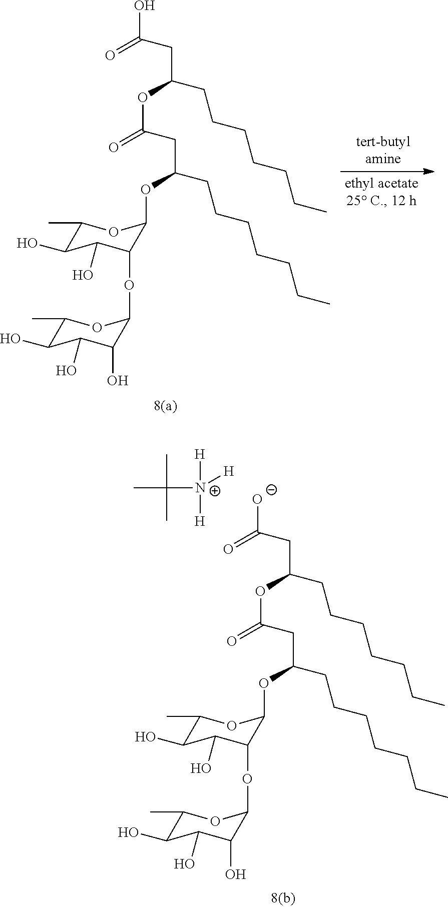

A flame-dried 10 mL round bottom flask under ambient conditions (e.g., room temperature at atmospheric pressure) was charged with ethyl acetate (4 mL) and 1 equiv (0.5 g, 1 mmol based on MW 650) of a biosynthesized (R,R) α-L-rhamnosyl-rhamnosyl-β-hydroxyalkanoyl-β-hydroxyalkanoates (diRL) 8(a) harvested and purified from Pseudomonas aeruginaos ATCC 9027. To this solution was added dropwise 1 equiv of tert-butyl amine (0.09 mL, 1 mmol), and the reaction mixture was stirred at room temperature for 12 h. The mixture was then concentrated in vacuo to yield the desired glyonic liquid 8(b) as an amorphous pale yellow solid in quantitative yield. The conductivity of 8(b) with small amounts of hydrating water ranged from 12 mS/cm to 16 mS/cm at temperatures between 25° C. and at 70° C., ¹H NMR (500 MHz, methanol-d₄) δ 5.35 (d, 1H), 4.89 (d, 2H), 4.13 (d, 1H), 3.97 (dd, 1H), 3.80 (dd, 1H), 3.73-3.63 (m, 5H), 3.38 (t, 1H), 3.24 (d, 1H), 2.56-2.34 (m, 4H), 1.62-1.52 (m, 4H), 1.35 (s, 9H), 1.33-1.28 (m, 20H), 1.24 (dd, 6H), 0.90 (td, 6H). ¹³C NMR (125 Hz, methanol-d₄) δ 173.24, 103.43, 97.95, 83.11, 74.65, 74.39, 74.25, 73.93, 72.30, 72.01, 71.67, 70.25, 70.14, 52.38, 44.81, 41.20, 38.86, 36.10, 34.06, 32.93, 30.81, 30.57, 30.31, 28.66, 26.31, 25.57, 24.70, 23.66, 19.27, 15.95.

### Synthesis of Rha-C₁₀-C₁₀—Choline Chloride (1:1) Type III DES

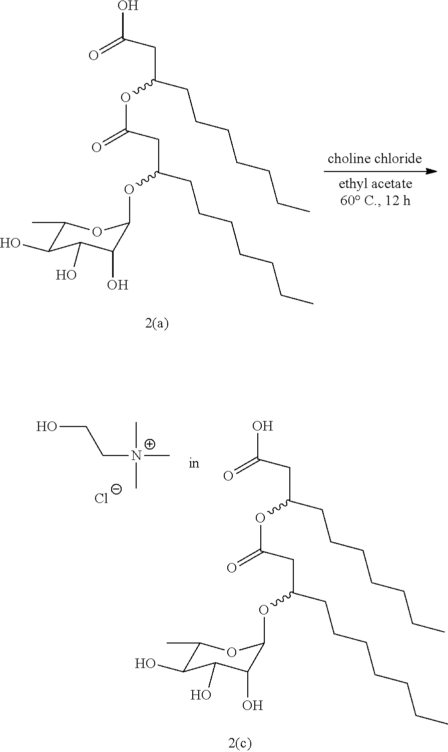

To a flame-dried 10 mL round bottom flask under ambient conditions was added 1 equiv of a diastereomeric mixture of α-L-rhamnopyranosyl-β-hydroxydecanoyl-β-hydroxydecanoate (2(a)) comprised of approximately equivalent amounts of the (R,R), (R,S), (S,R), and (S,S) diastereomers (0.6 g, 1.2 mmol) and ethyl acetate (5 mL). To the resulting solution was added 1 equiv of choline chloride (0.17 g, 1.2 mmol), and the reaction mixture was stirred in a sand bath at 60° C. for 12 h. The mixture was then concentrated in vacuo to yield the desired Type III deep eutectic solvent 2(c) as a homogeneous, colorless liquid in quantitative yield. The conductivity of 2(c) at 24° C. was 0.5 mS/cm. With small amounts of hydrating water, the conductivity was 1.1 mS/cm at 24° C. ¹H NMR (500 MHz, methanol-d₄) δ 4.85-4.77 (m, 1H), 4.05-3.98 (m, 1H), 3.69-3.58 (m, 4H), 3.53-3.48 (m, 2H), 3.36 (d, 3H), 3.23 (s, 9H), 2.65-2.45 (m, 4H), 1.67-1.54 (m, 2H), 1.37-1.28 (m, 22H), 0.92 (td, 6H). ¹³C NMR (125 MHz, methanol-d₄) δ 172.82, 172.80, 170.81, 170.79, 109.95, 102.66, 99.63, 99.26, 74.76, 74.58, 74.45, 74.33, 73.18, 71.23, 70.98, 70.46, 68.94, 68.90, 67.70, 67.67, 67.65, 55.72, 53.74, 53.34, 53.31, 48.50, 40.22, 40.03, 39.03, 38.46, 34.86, 33.68, 33.44, 32.97, 31.69, 31.64, 31.62, 31.60, 29.88, 29.33, 29.10, 29.02, 28.93, 25.50, 25.01, 24.92, 24.74, 24.57, 22.39, 22.38, 22.36, 16.61, 13.11.

### Synthesis of Rha-C₁₀—Choline Chloride (1:1) Type III DES

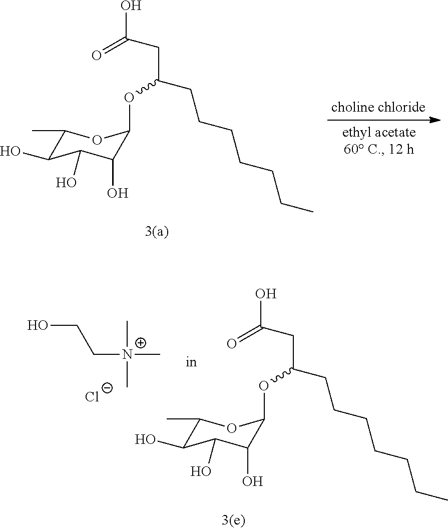

To a flame-dried 10 mL round bottom flask under ambient conditions was added 1 equiv of a diastereomeric mixture of α-L-rhamnose 3-hydroxydecanoic acid (3(a)) comprised of approximately equivalent amounts of the R and S diastereomers (0.5 g, 1.5 mmol) and ethyl acetate (5 mL). To the resulting solution was added 1 equiv of choline chloride (0.20 g, 1.5 mmol), and the reaction mixture was stirred in a sand bath at 60° C. for 12 h. The mixture was then concentrated in vacuo to yield the desired Type III deep eutectic solvent 3(e) as a homogeneous, colorless liquid in quantitative yield. ¹H NMR (500 MHz, methanol-d₄) δ 4.86-4.84 (m, 1H), 3.99 (dq, 3H), 3.77-3.58 (m, 3H), 3.52-3.47 (m, 3H), 3.21 (s, 9H), 2.53-2.22 (m, 2H), 1.61-1.47 (m, 12H), 1.23 (dd, 3H), 0.89 (t, 3H). ¹³C NMR (125 MHz, methanol-d₄) δ 179.43, 179.21, 101.44, 100.05, 77.86, 76.56, 74.25, 74.02, 73.25, 72.41, 70.07, 69.89, 69.05, 69.00, 57.07, 54.74, 54.70, 54.67, 52.66, 44.72, 44.33, 37.84, 37.28, 34.50, 33.05, 33.01, 30.89, 30.80, 30.43, 27.74, 26.57, 26.02, 24.44, 19.72, 15.11.

### Synthesis of Cellobiose-C₁₀-C₁₀—Choline Chloride (1:1) Type III DES

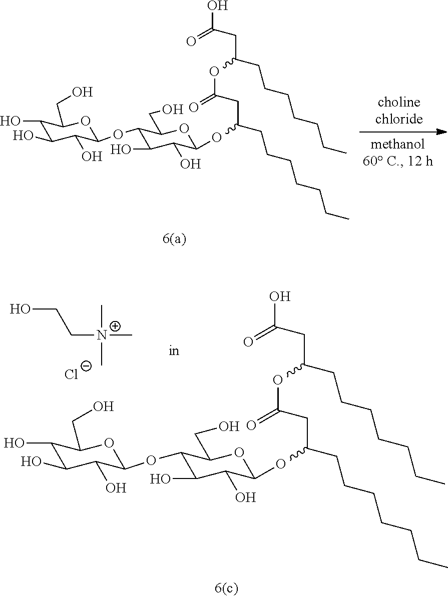

To a flame-dried 10 mL round bottom as under ambient conditions is added 1 equiv of a diastereomeric mixture of 4-O-β-D-glucopyranosyl-D-glucose-β-hydroxydecanoyl-β-hydroxydecanoate (6(a)) comprised of approximately equivalent amounts of the (R,R), (R,S), (S,R), and (S,S) diastereomers (0.5 g, 0.73 mmol) and methanol (4 mL). To the resulting solution is added 1 equiv of choline chloride (0.10 g, 0.73 mmol), and the reaction mixture is stirred in a sand bath at 60° C. for 12 h. The mixture is then concentrated in vacuo to yield the desired Type III deep eutectic solvent 6(c) as a homogeneous liquid in quantitative yield.

### Synthesis of a Maltotriose-C₁₀C₁₀—Choline Chloride (1:1) Type III DES

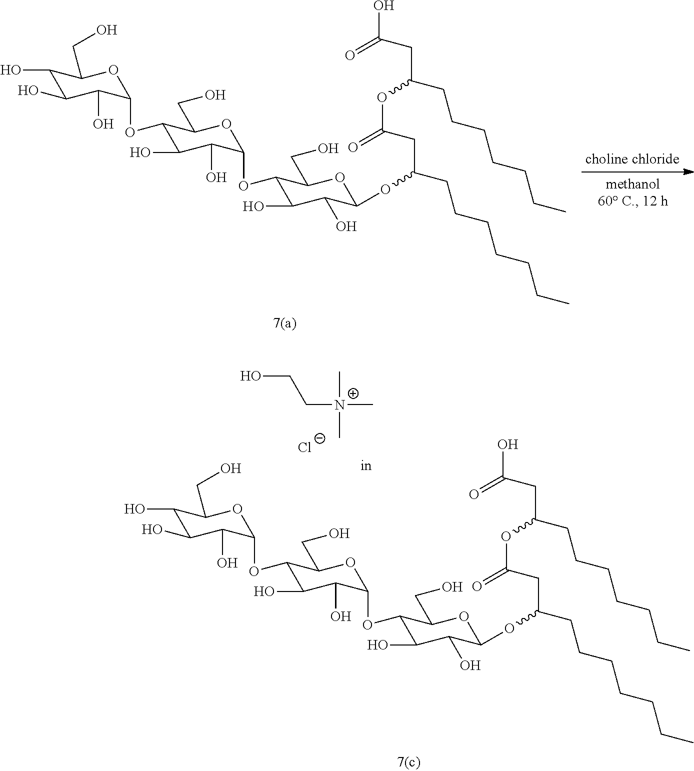

To a flame-dried 10 mL round bottom flask under ambient conditions is added 1 equiv of a diastereomeric mixture of O-α-DD-glucopyranosyl-(1→4)-O-α-D-glucopyranosyl-(1→4)-D-glucose-β-hydroxydecanoyl-β-hydroxydecanoate (7(a)) comprised of approximately equivalent amounts of the (R,R), (R,S), (S,R), and (S,S) diastereomers (0.5 g, 0.60 mmol) and methanol (4 mL). To the resulting solution is added 1 equiv of choline chloride (0.08 g, 0.60 mmol), and the reaction mixture is stirred in a sand bath at 60° C. for 12 h. The mixture is then concentrated in vacuo to yield the desired Type III deep eutectic solvent 7(c) as a homogeneous liquid in quantitative yield.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

1. An ionic liquid comprising a carbohydrate anionic moiety and a cationic counter-ion (Q⁺), wherein said carbohydrate anionic moiety is of the formula:
[G-L]⁻ I
wherein
G is selected from the group consisting of a monosaccharide, a disaccharide, a trisaccharide, and a derivative thereof; and
L is a moiety selected from the group consisting of: 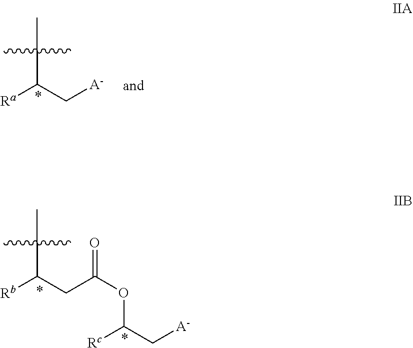
wherein
each of R^{a}, R^{b}, and R^{c} is independently hydrogen, C₁₋₁₈ alkyl, or C₂₋₂₀ mono- or di-unsaturated alkenyl;
A⁻ is —CO₂⁻, —PO₃H⁻, or —SO₃⁻; and
each of * marked carbon atom is independently a chiral center when said carbon atom has four different groups attached thereto.

2. The ionic liquid of claim 1, wherein said cationic counter-ion is selected from the group consisting of H⁺, imidazolium, pyridinium, pyrrolium, ammonium, iminium, phosphonium, sulfonium, an ester derivative of amino acid, choline, an ester derivative of betaine, and acetylcholine.

3. The ionic liquid of claim 1, wherein G comprises glucose, galactose, rhamnose, arabinose, xylose, fucose, glucosamine, lactose, maltose, melibiose, cellobiose, rutinose, maltotriose or a combination thereof.

4-8. (canceled)

9. The ionic liquid of claim 1, wherein L is a moiety of the formula: 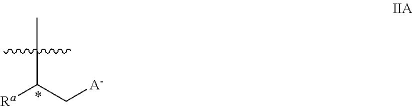
wherein A⁻, R^{a}, and * are as defined in claim 1.

10. The ionic liquid of claim 9, wherein A⁻ is —COO⁻, R^{a} is a C₇-, C₉-, or C₁₁ alkyl, or C₉- or C₁₁-monounsaturated alkenyl, and * is an (R)-isomer.

11. The ionic liquid of claim 1, wherein L is a moiety of the formula: 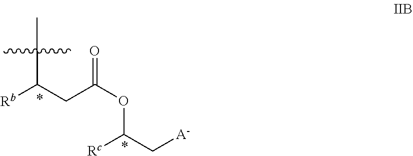
wherein A⁻, R^{b}, R^{c}, and * are as defined in claim 1.

12. The ionic liquid of claim 11, wherein A⁻ is —COO⁻, and each of R^{b} and R^{c} is independently a C₇-, C₉-, or C₁₁-alkyl, or C₉- or C₁₁-monounsaturated alkenyl.

13. The ionic liquid of claim 12, wherein each of R^{b} and R^{c} is independently H, or C₁-, C₃-, C₅-, C₇-, C₉-, C₁₁-, C₁₃-alkyl, or C₉- or C₁₁-monounsaturated alkenyl.

14. The ionic liquid of claim 1, wherein the melting point temperature of said ionic liquid is at most about 50° C.

15. The ionic liquid of claim 1, wherein said ionic liquid is a conductive non-crystalline semi-solid or solid at room temperature with a melting point temperature of at least 20° C.

16. A superionic proton conductive material comprising an ionic liquid of claim 1.

17. The ionic liquid of claim 1, wherein said ionic liquid is solvated or hydrated.

18. A superionic proton conductor comprising an ionic liquid of claim 17.

19. A fuel cell comprising an ionic liquid of claim 1.

20. The fuel cell of claim 19, wherein said ionic liquid is solvated or hydrated.

21. The fuel cell of claim 19, wherein said fuel cell is a battery.

22. The fuel cell of claim 19, wherein said fuel cell is a sodium or a lithium battery.

23. The ionic liquid of claim 1, wherein said ionic liquid is of the formula Qₓ⁺.[G-L]⁻, where x ranges from 0.5 to 1.

24. A method for separating a gas from a gaseous mixture comprising a first gas and at least one other gas that is different from said first gas, said method comprising contacting the gaseous mixture with a composition comprising an ionic liquid of claim 1 to dissolve at least a portion of said first gas in said composition, thereby separating at least a portion of said first gas from said gaseous mixture.

25. The method of claim 24, wherein said first gas comprises carbon dioxide, hydrogen sulfide, sulfur dioxide, methane, ethane, ethylene, propane, propylene, butane, 1-butene, oxygen, hydrogen, carbon monoxide, ammonia, water, Ar, Xe, CF₄, BF₃, AsH₃, or PH₃.
